(19) United States
(12) Patent Application Publication
(10) Pub. No.: US 2017/0252351 A1
(43) Pub. Date: Sep. 7, 2017

(54) Antibacterial Compositions

(71) Applicant: WOCKHARDT LIMITED, Chikathana, Auragabad (IN)

(72) Inventors: Mahesh Vithalbhai PATEL, Aurangabad (IN); Sachin BHAGWAT, Aurangabad -5, MAHARASHTRA (IN); Swapna Shripad TAKALKAR, Aurangabad -5 MAHARASHTRA (IN); Amol KULKARNI, Parbhani - 1,MAHARASHTRA (IN); Jaykumar Satwaji SATAV, Hingoli (IN); Snehal Rameshwar PALWE, Buldhana (IN); Hemant Narendra KHANDE, Nashik (IN)

(73) Assignee: WOCKHARDT LIMITED, Chikathana, Auragabad (IN)

(21) Appl. No.: 15/518,255

(22) PCT Filed: Jan. 22, 2016
(86) PCT No.: PCT/IB2016/050315

§ 371 Date: Apr. 11, 2017

(30) Foreign Application Priority Data
Jan. 24, 2015 (IN) 250/MUM/2015

(51) Int. Cl. A61K 31/546; A61K 9/20; A61K 9/08; A61K 9/00; A61K 31/424; A61K 9/14

(52) U.S. Cl. CPC A61K 31/546; A61K 31/424; A61K 9/14; A61K 9/08; A61K 9/0053; A61K 9/20

(57) ABSTRACT

Pharmaceutical compositions comprising ceftibuten or a pharmaceutically acceptable derivative thereof, and clavulanic acid or a pharmaceutically acceptable derivative thereof are disclosed.

### PRIORITY APPLICATION(S)

This application claims priority to Indian Patent Application No. 250/MUM/2015 filed on Jan. 24, 2015, the disclosures of which are incorporated herein by reference in its entirety as if fully rewritten herein.

### FIELD OF THE INVENTION

The invention relates to antibacterial compositions and methods for treating or preventing bacterial infections. Particularly, pharmaceutical compositions comprising: (a) ceftibuten or a pharmaceutically acceptable derivative thereof, and (b) clavulanic acid or a pharmaceutically acceptable derivative thereof, are disclosed.

### BACKGROUND OF THE INVENTION

Bacterial infections continue to remain one of the major causes contributing towards human diseases. One of the key challenges in treatment of bacterial infections is the ability of bacteria to develop resistance to one or more antibacterial agents over time. Examples of such bacteria that have developed resistance to typical antibacterial agents include: Penicillin-resistant Streptococcus pneumoniae, Vancomycin-resistant Enterococci, and Methicillin-resistant Staphylococcus aureus. The problem of emerging drug-resistance in bacteria is often tackled by switching to newer antibacterial agents, which can be more expensive and sometimes more toxic. Additionally, this may not be a permanent solution as the bacteria often develop resistance to the newer antibacterial agents as well in due course. In general, bacteria are particularly efficient in developing resistance, because of their ability to multiply very rapidly and pass on the resistance genes as they replicate.

The persistent exposure of bacterial strains to a multitude of beta-lactam antibacterial agents has led to overproduction and mutation of beta-lactamases. These new extended spectrum beta-lactamases (ESBL) are capable of hydrolyzing penicillins, cephalosporins, monobactams and even carbapenems. Non enzymatic resistance mechanisms such as under-expression of outer membrane porins and/or efflux pumps have also caused resistance towards carbapenems. Such a wide spread resistance to many of the existing beta-lactam antibacterial agents, either used alone or in combination with other agents, is posing challenges in treating serious bacterial infections.

Therefore, there is a need for development of agents capable of acting against ESBLs, which are currently resistant to available combination of beta-lactam and beta-lactamase inhibitors. In particular, there is severe dearth of oral antibacterial agents that can provide good therapeutic efficacy for infections caused by multi-drug resistant gram negative pathogens (particularly those belonging to Enterobactereaceae). International Patent Application No. PCT/KR2012/009391 disclose compositions comprising combinations of cephalosporins selected from 1^{st} generation, 2^{nd} generation, 4^{th} generation, and 5^{th} generation cephalosporins with beta-lactamase inhibitors. International Patent Application No. PCT/TR2014/000041 discloses formulation containing combination of cefixime and clavulanic acid. Current beta-lactam-beta-lactamase inhibitor combinations show therapeutically relevant activity against only Class A beta-lactamase expressing pathogens that confer resistance to most cephalosporins. However, these combination products do not provide therapeutic coverage for organisms expressing other resistance mechanisms such as Klebsiella pneumonia carbapenemase (KPC) or outer membrane porin down (OMP) regulation that confer resistance to carbapenems. Surprisingly, it has been found that a pharmaceutical composition comprising ceftibuten and clavulanic acid exhibits unexpectedly improved antibacterial efficacy, even against highly resistant gram negative bacteria.

### SUMMARY OF THE INVENTION

Accordingly, there are provided pharmaceutical compositions comprising synergistically effective amount of: (a) ceftibuten or a pharmaceutically acceptable derivative thereof, and (b) clavulanic acid or a pharmaceutically acceptable derivative thereof.

In one general aspect, there is provided a pharmaceutical composition comprising synergistically effective amount of: (a) ceftibuten or a pharmaceutically acceptable derivative thereof, and (b) clavulanic acid or a pharmaceutically acceptable derivative thereof; wherein the weight ratio of clavulanic acid or a pharmaceutically acceptable derivative thereof, to ceftibuten or a pharmaceutically acceptable derivative thereof in the composition is from about 1:8 to about 8:1.

In one general aspect, there is provided a pharmaceutical composition comprising synergistically effective amount of: (a) ceftibuten or a pharmaceutically acceptable derivative thereof, and (b) clavulanic acid or a pharmaceutically acceptable derivative thereof; wherein clavulanic acid or a pharmaceutically acceptable derivative thereof in the composition is present in an amount from about 0.25 gram to about 4 gram per gram of ceftibuten or a pharmaceutically acceptable derivative thereof.

In another general aspect, there is provided a method for treating or preventing a bacterial infection in a subject, said method comprising administering to said subject a pharmaceutical composition comprising synergistically effective amount of: (a) ceftibuten or a pharmaceutically acceptable derivative thereof, and (b) clavulanic acid or a pharmaceutically acceptable derivative thereof.

In another general aspect, there is provided a method for treating or preventing a bacterial infection in a subject, said method comprising administering to said subject synergistically effective amount of: (a) ceftibuten or a pharmaceutically acceptable derivative thereof, and (b) clavulanic acid or a pharmaceutically acceptable derivative thereof.

In another general aspect, there is provided a method for treating or preventing a bacterial infection in a subject, said method comprising administering to said subject synergistically effective amount of: (a) ceftibuten or a pharmaceutically acceptable derivative thereof, and (b) clavulanic acid or a pharmaceutically acceptable derivative thereof; wherein the weight ratio of clavulanic acid or a pharmaceutically acceptable derivative thereof, to ceftibuten or a pharmaceutically acceptable derivative thereof in the composition is from about 1:8 to about 8:1.

In another general aspect, there is provided a method for treating or preventing a bacterial infection in a subject, said method comprising administering to said subject synergistically effective amount of: (a) ceftibuten or a pharmaceutically acceptable derivative thereof, and (b) clavulanic acid or a pharmaceutically acceptable derivative thereof; wherein amount of clavulanic acid or a pharmaceutically acceptable derivative thereof administered is from about 0.25 gram to about 4 gram per gram of ceftibuten or a pharmaceutically acceptable derivative thereof.

In yet another general aspect, there is provided a method for increasing antibacterial effectiveness of ceftibuten or a pharmaceutically acceptable derivative thereof in a subject, said method comprising co-administering the ceftibuten or a pharmaceutically acceptable derivative thereof, with clavulanic acid or a pharmaceutically acceptable derivative thereof.

The details of one or more embodiments of the invention are set forth in the description below. Other features, objects and advantages of the invention will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, which would occur to one skilled in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety.

The inventors have surprisingly discovered that a pharmaceutical composition comprising: (a) ceftibuten or a pharmaceutically acceptable derivative thereof, and (b) clavulanic acid or a pharmaceutically acceptable derivative thereof, wherein when ceftibuten and clavulanic acid are present in specific amounts, exhibits unexpectedly improved antibacterial efficacy, even against highly resistant gram negative bacteria, including those producing extended spectrum beta-lactamase enzymes (ESBLs).

The term “infection” or “bacterial infection” as used herein includes presence of bacteria, in or on a subject, which, if its growth were inhibited, would result in a benefit to the subject. As such, the term “infection” in addition to referring to the presence of bacteria also refers to presence of other floras, which are not desirable. The term “infection” includes infection caused by bacteria.

The term “treat”, “treating” or “treatment” as used herein refers to administration of a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to administering treatment to a subject already suffering from infection. The terms “treat”, “treating” or “treatment” as used herein also refer to administering compositions, or one or more of pharmaceutically active ingredients discussed herein, with or without additional pharmaceutically active or inert ingredients, in order to: (i) reduce or eliminate either a bacterial infection, or one or more symptoms of a bacterial infection, or (ii) retard progression of a bacterial infection, or one or more symptoms of a bacterial infection, or (iii) reduce severity of a bacterial infection, or one or more symptoms of a bacterial infection, or (iv) suppress clinical manifestation of a bacterial infection, or (v) suppress manifestation of adverse symptoms of a bacterial infection.

The terms “pharmaceutically effective amount” or “therapeutically effective amount” or “effective amount” as used herein refer to an amount, which has a therapeutic effect or is the amount required to produce a therapeutic effect in a subject. For example, a “therapeutically effective amount” or “pharmaceutically effective amount” or “effective amount” of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). Such effective amount depends on several factors, including but not limited to, the microorganism (e.g. bacteria) involved, characteristics of the subject (for example height, weight, sex, age and medical history), severity of infection and particular type of the antibacterial agent used. For prophylactic treatments, a prophylactically effective amount is that amount which would be effective in preventing the bacterial infection.

The term “administration” or “administering” refers to and includes delivery of a composition, or one or more pharmaceutically active or inert ingredients to a subject, including for example, by any appropriate method, which serves to deliver the composition or its active ingredients, one or more pharmaceutically active or inert ingredients to the site of infection. The method of administration may vary depending on various factors, such as for example, the components of the pharmaceutical composition or type/nature of the pharmaceutically active or inert ingredients, site of the potential or actual infection, the microorganism (e.g. bacteria) involved, severity of the infection, age and physical condition of the subject and a like. Some non-limiting examples of ways to administer a composition or a pharmaceutically active ingredient to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop and mouthwash. In case of a pharmaceutical composition comprising more than one ingredients (active or inert), one of the ways of administering such composition is by admixing the ingredients (e.g. in the form of a suitable unit dosage form such as tablet, capsule, solution, powder or a like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “growth” as used herein refers to a growth of one or more microorganisms and includes reproduction or population expansion of the microorganism (e.g. bacteria). The term “growth” also includes maintenance of on-going metabolic processes of the microorganism, including the processes that keep the microorganism alive.

The term, “effectiveness” as used herein refers to ability of a treatment, or a composition, or one or more pharmaceutically active ingredients to produce a desired biological effect in a subject. For example, the term “antibacterial effectiveness” of a composition or of an antibacterial agent refers to the ability of the composition or the antibacterial agent to prevent or treat bacterial infection in a subject.

The term “synergistic” or “synergy” as used herein refers to the interaction of two or more agents so that their combined effect is greater than their individual effects.

The term “synergistically effective amounts” as used herein refers to amounts of each active component in the treatment which are effective in producing more than additive effect of each component. The term “synergistically effective amounts” also includes the amounts of two or more active agents that provides a synergistic effect.

The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

The term “beta-lactamase” or “beta-lactamase enzyme” as used herein refers to any enzyme or protein or any other substance that breaks down a beta-lactam ring. The term “beta-lactamase” includes enzymes that are produced by bacteria and have the ability to hydrolyse the beta-lactam ring in a beta-lactam compound, either partially or completely.

The term “extended spectrum beta-lactamase” (ESBL) as used herein includes those beta-lactamase enzymes, which are capable of conferring bacterial resistance to various beta-lactam antibacterial agents such as penicillins, cephalosporins, aztreonam and the like.

The term “beta-lactamase inhibitor” as used herein refers to a compound capable of inhibiting activity of one or more beta-lactamase enzymes, either partially or completely.

The term “pharmaceutically inert ingredient” or “carrier” or “excipient” refers to and includes compounds or materials used to facilitate administration of one or more compounds (or one or more active ingredients), for example, to increase the solubility of the compound. Typical, non-limiting examples of solid carriers include starch, lactose, dicalcium phosphate, sucrose, and kaolin. Typical, non-limiting examples of liquid carriers include sterile water, saline, buffers, non-ionic surfactants, and edible oils. In addition, various adjuvants commonly used in the art may also be included. These and other such compounds are described in literature, e.g., in the Merck Index (Merck & Company, Rahway, N.J.). Considerations for inclusion of various components in pharmaceutical compositions are described, e.g., in Gilman et al. (Goodman and Gilman's: The Pharmacological Basis of Therapeutics, 8th Ed., Pergamon Press, 1990), which is incorporated herein by reference in its entirety.

The term “subject” as used herein refers to vertebrate or invertebrate, including a mammal. The term “subject” also includes vertebrate or invertebrate, including a mammal, which is in need of a therapeutic or prophylactic treatment, such as antibacterial treatment. The term “subject” includes human, animal, a bird, a fish, or an amphibian. Typical, non-limiting examples of a “subject” include humans, cats, dogs, horses, sheep, bovine cows, pigs, lambs, rats, mice and guinea pigs.

The term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the parent compound. For example, the term “antibacterial agent or a pharmaceutically acceptable derivative thereof” includes all derivatives of the antibacterial agent (such as salts, pro-drugs, metabolites, esters, ethers, hydrates, polymorphs, solvates, complexes, and adducts) which, upon administration to a subject, are capable of providing (directly or indirectly) the antibacterial agent.

The term “pharmaceutically acceptable salt” as used herein refers to one or more salts of a given compound which possesses desired pharmacological activity of the free compound and which is neither biologically nor otherwise undesirable. In general, the term “pharmaceutically acceptable salts” refer to salts that are suitable for use in contact with the tissues of human and animals without undue toxicity, irritation, allergic response and the like, and are commensurate with a reasonable benefit/risk ratio. Pharmaceutically acceptable salts are well known in the art. For example, S. M. Berge, et al. (J. Pharmaceutical Sciences, 66; 1-19, 1977), incorporated herein by reference in its entirety, describes various pharmaceutically acceptable salts in details.

In one general aspect, there are provided pharmaceutical compositions comprising synergistically effective amount of: (a) ceftibuten or a pharmaceutically acceptable derivative thereof, and (b) clavulanic acid or a pharmaceutically acceptable derivative thereof.

A person of skills in the art would appreciate that various compounds described herein (including, for example ceftibuten and clavulanic acid) can exist and are often used as their pharmaceutically acceptable derivatives (such as salts, pro-drugs, metabolites, esters, ethers, hydrates, polymorphs, solvates, complexes, and adducts). The clavulanic acid may also be used in the form of its pharmaceutically acceptable salts such as sodium, potassium or any other pharmaceutically acceptable salt. Typical, non-limiting examples of suitable pharmaceutically acceptable salts of clavulanic acid include sodium clavulanate, potassium clavulanate and the like. The ceftibuten may be used in its free form or in the form of its pharmaceutically acceptable derivatives. Typical, non-limiting examples of pharmaceutically acceptable derivatives of ceftibuten include ceftibuten dihydrate.

In some embodiments, there is provided a pharmaceutical composition comprising synergistically effective amount of: (a) ceftibuten dihydrate and (b) potassium clavulanate.

Individual amounts of the ceftibuten or a pharmaceutically acceptable derivative thereof, and clavulanic acid or a pharmaceutically acceptable derivative thereof in the composition may vary depending on clinical requirements. The specified amount of ceftibuten and clavulanic acid is calculated on the basis of their equivalent free forms.

In one general aspect, there are provided pharmaceutical compositions comprising synergistically effective amount of: (a) ceftibuten or a pharmaceutically acceptable derivative thereof, and (b) clavulanic acid or a pharmaceutically acceptable derivative thereof; wherein the weight ratio of clavulanic acid or a pharmaceutically effective amount of, to ceftibuten or a pharmaceutically acceptable derivative in the composition is from about 1:8 to about 8:1.

In some embodiments, there is provided a pharmaceutical composition consisting of synergistically effective amount of: (a) ceftibuten or a pharmaceutically acceptable derivative thereof, and (b) clavulanic acid or a pharmaceutically acceptable derivative thereof as active ingredients; wherein the weight ratio of clavulanic acid or a pharmaceutically acceptable derivative thereof, to ceftibuten or a pharmaceutically acceptable derivative thereof in the composition is from about 1:8 to about 8:1. The pharmaceutical compositions may further comprise one or more pharmaceutically inert ingredients.

In one general aspect, there are provided pharmaceutical compositions comprising synergistically effective amount of: (a) ceftibuten or a pharmaceutically acceptable derivative thereof, and (b) clavulanic acid or a pharmaceutically acceptable derivative thereof; wherein the weight ratio of clavulanic acid or a pharmaceutically effective amount of, to ceftibuten or a pharmaceutically acceptable derivative in the composition is 1:8, 1:4, 1:2, 1:1, 5:8, 5:16, 2:1, 4:1 or 8:1.

In some embodiments, there are provided pharmaceutical compositions comprising synergistically effective amount of: (a) ceftibuten or a pharmaceutically acceptable derivative thereof, and (b) clavulanic acid or a pharmaceutically acceptable derivative thereof; wherein clavulanic acid or a pharmaceutically acceptable derivative thereof in the composition is present in an amount from about 0.25 gram to about 4 gram per gram of ceftibuten or a pharmaceutically acceptable derivative thereof.

In some other embodiments, ceftibuten or a pharmaceutically acceptable derivative thereof is present in the composition in an amount from about 0.01 gram to about 10 gram. In some other embodiments, clavulanic acid or a pharmaceutically acceptable derivative thereof, is present in the composition in an amount from about 0.01 gram to about 10 gram.

In some embodiments, the pharmaceutical composition according to invention comprises about 0.2 gram of ceftibuten or a pharmaceutically acceptable derivative thereof and about 0.4 gram of clavulanic acid or a pharmaceutically acceptable derivative thereof.

In some embodiments, the pharmaceutical composition according to invention comprises about 0.2 gram of ceftibuten or a pharmaceutically acceptable derivative thereof and about 0.2 gram of clavulanic acid or a pharmaceutically acceptable derivative thereof.

In some embodiments, the pharmaceutical composition according to invention comprises about 0.2 gram of ceftibuten or a pharmaceutically acceptable derivative thereof and about 0.125 gram of clavulanic acid or a pharmaceutically acceptable derivative thereof.

In some embodiments, the pharmaceutical composition according to invention comprises about 0.2 gram of ceftibuten or a pharmaceutically acceptable derivative thereof and about 0.0625 gram of clavulanic acid or a pharmaceutically acceptable derivative thereof.

In some embodiments, the pharmaceutical composition according to invention comprises about 0.4 gram of ceftibuten or a pharmaceutically acceptable derivative thereof and about 0.8 gram of clavulanic acid or a pharmaceutically acceptable derivative thereof.

In some embodiments, the pharmaceutical composition according to invention comprises about 0.4 gram of ceftibuten or a pharmaceutically acceptable derivative thereof and about 0.4 gram of clavulanic acid or a pharmaceutically acceptable derivative thereof.

In some embodiments, the pharmaceutical composition according to invention comprises about 0.4 gram of ceftibuten or a pharmaceutically acceptable derivative thereof and about 0.2 gram of clavulanic acid or a pharmaceutically acceptable derivative thereof.

In some embodiments, the pharmaceutical composition according to invention comprises about 0.4 gram of ceftibuten or a pharmaceutically acceptable derivative thereof and about 0.125 gram of clavulanic acid or a pharmaceutically acceptable derivative thereof.

In some embodiments, the pharmaceutical composition according to invention comprises about 0.4 gram of ceftibuten or a pharmaceutically acceptable derivative thereof and about 0.0625 gram of clavulanic acid or a pharmaceutically acceptable derivative thereof.

In some embodiments, the pharmaceutical composition according to invention comprises about 0.5 gram of ceftibuten or a pharmaceutically acceptable derivative thereof and about 0.2 gram of clavulanic acid or a pharmaceutically acceptable derivative thereof.

In some embodiments, the pharmaceutical composition according to invention comprises about 0.5 gram of ceftibuten or a pharmaceutically acceptable derivative thereof and about 0.125 gram of clavulanic acid or a pharmaceutically acceptable derivative thereof.

In some embodiments, the pharmaceutical composition according to invention comprises about 0.5 gram of ceftibuten or a pharmaceutically acceptable derivative thereof and about 0.0625 gram of clavulanic acid or a pharmaceutically acceptable derivative thereof.

The pharmaceutical compositions according to the invention may include one or more pharmaceutically acceptable inactive ingredients such as carriers or excipients or the like. Typical, non-limiting examples of such carriers or excipients include mannitol, lactose, starch, magnesium stearate, sodium saccharine, talcum, cellulose, sodium crosscarmellose, glucose, gelatine, sucrose, magnesium carbonate, wetting agents, emulsifying agents, solubilizing agents, buffering agents, lubricants, preservatives, stabilizing agents, binding agents and the like.

The pharmaceutical compositions or the active ingredients according to the present invention may be formulated into a variety of dosage forms, such as solid, semi-solid, liquid and aerosol dosage forms. Typical, non-limiting examples of some dosage forms include tablets, capsules, powders, solutions, suspensions, suppositories, aerosols, granules, emulsions, syrups, elixirs and the like.

Depending on the requirement, the pharmaceutical compositions according to the invention may also be prepared and packaged in bulk form. Alternatively, the pharmaceutical compositions of the invention may be prepared and packaged in unit dosage form.

In some embodiments, pharmaceutical compositions according to the invention are in the form of a powder or a solution. In some other embodiments, pharmaceutical compositions according to the invention are present in the form of a powder or a solution that can be reconstituted by addition of a compatible reconstitution diluent prior to administration. In some other embodiments, pharmaceutical compositions according to the invention are in the form of a frozen composition that can be diluted with a compatible reconstitution diluent prior to administration. Typical, non-limiting example of suitable compatible reconstitution diluent includes water.

In some other embodiments, pharmaceutical compositions according to the invention are present in the form ready to use for oral or parenteral administration.

The pharmaceutical compositions to the invention may be formulated into a variety of solid oral dosage forms. Typical, non-limiting examples of some oral dosage forms include tablet, capsule, powder, discs, caplets, pellets, granules, granules in capsule, minitablets, minitablets in capsule, pellets in capsule and the like. In some embodiments, the compositions according to invention may also be formulated into other dosage form suitable for oral administration such as suspensions, emulsions, syrups, elixirs and the like.

The compositions according to the invention can be formulated into various dosage forms wherein the active ingredients and/or excipients may be present either together (e.g. as an admixture) or as separate components. When the various ingredients in the composition are formulated as a mixture, such compositions can be delivered by administering such a mixture to a subject using any suitable route of administration. Alternatively, pharmaceutical compositions according to the invention may also be formulated into a dosage form wherein one or more ingredients (such as active or inactive ingredients) are present as separate components. The composition or dosage form wherein the ingredients do not come as a mixture, but come as separate components, such composition/dosage form may be administered in several ways. In one possible way, the ingredients may be mixed in the desired proportions and the mixture is reconstituted in suitable reconstitution diluent and is then administered as required. Alternatively, the components or the ingredients (active or inert) may be separately administered (simultaneously or one after the other) in appropriate proportion so as to achieve the same or equivalent therapeutic level or effect as would have been achieved by administration of the equivalent mixture.

In some embodiments, pharmaceutical compositions according to the invention are formulated into a dosage form such that ceftibuten or a pharmaceutically acceptable derivative thereof, and clavulanic acid or a pharmaceutically acceptable derivative thereof are present in the composition as admixture or as separate components. In some other embodiments, pharmaceutical compositions according to the invention are formulated into a dosage form such that ceftibuten or a pharmaceutically acceptable derivative thereof, and clavulanic acid or a pharmaceutically acceptable derivative thereof, are present in the composition as separate components.

The pharmaceutical compositions according to the invention may include one or more pharmaceutically acceptable carriers or excipients or the like. Typical, non-limiting examples of such carriers or excipients include diluents, disintegrants, binders, wetting agents, emulsifying agents, solubilizing agents, buffering agents, glidants, lubricants, preservatives, stabilizing agents, flavoring agents and the like.

In some embodiments, there are provided pharmaceutical compositions comprising a ceftibuten or a pharmaceutically acceptable derivative thereof, and clavulanic acid or a pharmaceutically acceptable derivative thereof as an active ingredient and one or more excipients selected from diluent, disintegrant, binder, lubricant or glidant.

The pharmaceutical compositions to the invention may be formulated into a variety of solid oral dosage forms. Typical, non-limiting examples of some oral dosage forms include tablet, capsule, powder, discs, caplets, pellets, granules, granules in capsule, minitablets, minitablets in capsule, pellets in capsule and the like. In some embodiments, the compositions according to invention may also be formulated into other dosage form suitable for oral administration such as suspensions, emulsions, syrups, elixirs and the like.

In some embodiments, ceftibuten or a pharmaceutically acceptable derivative thereof present in the composition is in an amount within the range of from about 10% to about 90% by weight.

In some embodiments, clavulanic acid or a pharmaceutically acceptable derivative thereof present in the composition is in an amount within the range of from about 10% to about 90% by weight.

In some embodiments, diluent is present in an amount within the range of from about 1% to about 80% by weight. In some other embodiments, diluent is present in an amount within the range of from about 1% to about 50% by weight.

In some embodiments, disintegrant, if present, is present in an amount within the range of from about 0% to about 30% by weight. In some other embodiments, disintegrant is present in an amount within the range of from about 1% to about 15% by weight.

In some embodiments, binder, if present, is present in an amount within the range of from about 0% to about 30% by weight. In some other embodiments, binder is present in an amount within the range of from about 0.25% to about 10% by weight.

In some embodiments, glidant, if present, is present in an amount within the range of from about 0% to about 20% by weight. In some other embodiments, glidant is present in an amount within the range of from about 0.25% to about 10% by weight.

In some embodiments, lubricant, if present, is present in an amount within the range of from about 0% to about 20% by weight. In some other embodiments, lubricant is present in an amount within the range of from about 0.25% to about 5% by weight.

In some embodiments, the formulated tablets are coated with a suitable coating material dissolved in a suitable solvent. In some embodiments, coating is present in an amount within the range of from about 0.25% to about 5% by weight.

In some embodiments, there are provided pharmaceutical compositions comprising:

a ceftibuten or a pharmaceutically acceptable derivative thereof as an active ingredient in an amount between about 10% to about 90% by weight;

a clavulanic acid or a pharmaceutically acceptable derivative thereof as an active ingredient in an amount between about 10% to about 90% by weight;

at least one or more diluent in an amount between about 1% to about 50% by weight;

optionally one or more disintegrant in an amount between about 1% to about 15% by weight;

optionally one or more binder selected in an amount between about 0.25% to about 10% by weight;

optionally one or more lubricant in an amount between about 0.25% to about 5% by weight;

optionally one or more glidant in an amount between about 0.25% to about 10% by weight;

optionally film coating in an amount between about 0.25% to about 5% by weight.

Typical, non-limiting examples of diluents include microcrystalline cellulose, cellulose, lactose, starch, pregelatinized starch, corn starch, calcium carbonate, calcium sulfate, sugar, dextrates, sucrose, dextrin, fructose, dextrose, xylitol, polysaccharide, dibasic calcium phosphate dihydrate, tribasic calcium phosphate, calcium sulphate, kaolin, magnesium carbonate, magnesium oxide, maltodextrin, mannitol, polymethacrylates, potassium chloride, sodium chloride, sorbitol, and the like

Typical, non-limiting example of binders include acacia, alginic acid, carbomer (carbopol), carboxymethylcellulose sodium, corn starch, dextrin, ethyl cellulose, methyl cellulose, gelatin, guar gum, hydrogenated vegetable oil, hydroxyethyl cellulose, hydroxypropylcellulose, hydroxypropylmethylcellulose, liquid glucose, magnesium aluminium silicate, maltodextrin, methyl cellulose, cellulose acetate, polymethacrylates, povidone, polyvinyl alcohol, pregelatinized starch, sodium alginate, starch, carnuba wax, paraffin, spermaceti, polyethylenes, microcrystalline wax and the like

Typical, non-limiting examples of disintegrants include alginic acid, carboxymethylcellulose calcium, carboxymethylcellulose sodium, colloidal silicon dioxide, croscarmellose sodium, crospovidone, gura gum, low substituted hydroxypropyl cellulose, magnesium aluminium silicate, methyl cellulose, microcrystalline cellulose, polacrilin potassium, powdered cellulose, starch, pregelatinized starch, corn starch, potato starch, sodium alginate, sodium starch glycolate, and the like.

Typical, non-limiting examples of glidants include silicon dioxide, colloidal silicon dioxide, magnesium silicate, magnesium trisilicate, powdered cellulose, starch, talc, tribasic calcium phosphate and the like.

Typical non-limiting examples of lubricants include magnesium stearate, zinc stearate, calcium stearate, carnauba wax, palmitic acid, glyceryl monosterate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, myristic acid, talc, zinc stearate and the like.

In some embodiments, the compositions according to invention are coated with suitable coating polymers. Typical non-limiting examples of coating polymers include hydroxypropylmethyl cellulose, polyvinyl alcohol, ethyl cellulose, methacyrlic polymers, hydroxyproyl cellulose, starch and the like. In some embodiments, coating can optionally include a plasticizer. Typical, non-limiting examples of plasticizers include triacetin, diethyl phthalate, tributyl sebacate, polyethylene glycol, glycerin, triacetin, triethyl citrate and the like. In some embodiments, coating can also optionally include an anti-adherent or glidant. Typical, non-limiting examples of anti-adherent or glidant include talc, fumed silica, magnesium stearate and the like. In some other embodiments, coating can also optionally include an opacifier. Typical, non-limiting example of opacifier includes titanium dioxide and the like. In yet another embodiment, coating can also optionally include one or more colorants. In some embodiments, the compositions according to present invention are film coated with a suitable opadry coating material.

In some embodiments, the compositions according to the invention are formulated as tablets. Such tablets may be prepared using known techniques. In some embodiments, the compositions according to the invention are formulated as tablets by following dry granulation, wet granulation or direct compression techniques.

In one general aspect, pharmaceutical compositions according to the invention are used in treatment or prevention of a bacterial infection.

In another general aspect, there are provided methods for treating or preventing a bacterial infection in a subject, said methods comprising administering to said subject effective amount of a pharmaceutical composition according to the invention. In case of dosage forms wherein ceftibuten or a pharmaceutically acceptable derivative thereof, and clavulanic acid or a pharmaceutically acceptable derivative thereof are present in the composition as separate components; ceftibuten or a pharmaceutically acceptable derivative thereof may be administered before, after or simultaneously with the administration of clavulanic acid or a pharmaceutically acceptable derivative thereof. In some embodiments, the compositions according to the invention are administered orally or parenterally.

In another general aspect, there is provided a method for treating or preventing a bacterial infection in a subject, said method comprising administering to said subject synergistically effective amount of: (a) ceftibuten or a pharmaceutically acceptable derivative thereof, and (b) clavulanic acid or a pharmaceutically acceptable derivative thereof.

In some embodiments, there is provided a method for treating or preventing a bacterial infection in a subject, said method comprising administering to said subject: (a) ceftibuten or a pharmaceutically acceptable derivative thereof, and (b) clavulanic acid or a pharmaceutically acceptable derivative thereof; wherein the amount of clavulanic acid or a pharmaceutically effective amount of, and ceftibuten or a pharmaceutically acceptable derivative administered are in weight ratio of about 1:8 to about 8:1. In some embodiments, the amount of clavulanic acid or a pharmaceutically effective amount of, and ceftibuten or a pharmaceutically acceptable derivative administered are in weight ratio of about 1:8, 1:4, 1:2, 1:1, 5:8, 5:16, 2:1, 4:1 or 8:1.

In some embodiments, there is provided a method for treating or preventing a bacterial infection in a subject, said method comprising administering to said subject: (a) ceftibuten or a pharmaceutically acceptable derivative thereof, and (b) clavulanic acid or a pharmaceutically acceptable derivative thereof; wherein amount of clavulanic acid or a pharmaceutically acceptable derivative thereof administered is from about 0.25 gram to about 4 gram per gram of ceftibuten or a pharmaceutically acceptable derivative thereof.

In some embodiments, there is provided a method for treating or preventing a bacterial infection in a subject, said method comprising administering to said subject: (a) ceftibuten or a pharmaceutically acceptable derivative thereof, and (b) clavulanic acid or a pharmaceutically acceptable derivative thereof, in any of the following amounts:

(i) about 0.2 gram of ceftibuten or a pharmaceutically acceptable derivative thereof and about 0.4 gram of clavulanic acid or a pharmaceutically acceptable derivative thereof;

(ii) about 0.2 gram of ceftibuten or a pharmaceutically acceptable derivative thereof and about 0.2 gram of clavulanic acid or a pharmaceutically acceptable derivative thereof;

(iii) about 0.2 gram of ceftibuten or a pharmaceutically acceptable derivative thereof and about 0.125 gram of clavulanic acid or a pharmaceutically acceptable derivative thereof;

(iv) about 0.2 gram of ceftibuten or a pharmaceutically acceptable derivative thereof and about 0.0625 gram of clavulanic acid or a pharmaceutically acceptable derivative thereof;

(v) about 0.4 gram of ceftibuten or a pharmaceutically acceptable derivative thereof and about 0.8 gram of clavulanic acid or a pharmaceutically acceptable derivative thereof;

(vi) about 0.4 gram of ceftibuten or a pharmaceutically acceptable derivative thereof and about 0.4 gram of clavulanic acid or a pharmaceutically acceptable derivative thereof;

(vii) about 0.4 gram of ceftibuten or a pharmaceutically acceptable derivative thereof and about 0.2 gram of clavulanic acid or a pharmaceutically acceptable derivative thereof;

(viii) about 0.4 gram of ceftibuten or a pharmaceutically acceptable derivative thereof and about 0.125 gram of clavulanic acid or a pharmaceutically acceptable derivative thereof;

(ix) about 0.4 gram of ceftibuten or a pharmaceutically acceptable derivative thereof and about 0.0625 gram of clavulanic acid or a pharmaceutically acceptable derivative thereof;

(x) about 0.5 gram of ceftibuten or a pharmaceutically acceptable derivative thereof and about 0.2 gram of clavulanic acid or a pharmaceutically acceptable derivative thereof;

(xi) about 0.5 gram of ceftibuten or a pharmaceutically acceptable derivative thereof and about 0.125 gram of clavulanic acid or a pharmaceutically acceptable derivative thereof; or

(xii) about 0.5 gram of ceftibuten or a pharmaceutically acceptable derivative thereof and about 0.0625 gram of clavulanic acid or a pharmaceutically acceptable derivative thereof.

In some embodiments, in the methods according to the invention, ceftibuten or a pharmaceutically acceptable derivative thereof is administered in an amount from about 0.01 gram to about 10 gram.

In some other embodiments, in the methods according to the invention, clavulanic acid or a pharmaceutically acceptable derivative thereof is administered in an amount from about 0.01 gram to about 10 gram.

In some embodiments, in the methods according to the invention, ceftibuten or a pharmaceutically acceptable derivative thereof is administered before, after or simultaneously with the administration of clavulanic acid or a pharmaceutically acceptable derivative thereof. In some embodiments, ceftibuten or a pharmaceutically acceptable derivative thereof, and clavulanic acid or a pharmaceutically acceptable derivative thereof, are administered orally or parenterally.

In the methods according to the invention, the pharmaceutical composition and/or other pharmaceutically active ingredients disclosed herein may be administered by any appropriate method, which serves to deliver the composition, or its constituents, or the active ingredients to the desired site. The method of administration can vary depending on various factors, such as for example, the components of the pharmaceutical composition and the nature of the active ingredients, the site of the potential or actual infection, the microorganism (e.g. bacteria) involved, severity of infection, age and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In some embodiments, the compositions or one or more active ingredients according to the invention are administered orally or parenterally.

In some embodiments, there is provided a method for increasing antibacterial effectiveness of ceftibuten or a pharmaceutically acceptable derivative thereof in a subject, said method comprising co-administering ceftibuten or a pharmaceutically acceptable derivative thereof, with clavulanic acid or a pharmaceutically acceptable derivative thereof.

In some other embodiments, there is provided a method for increasing antibacterial effectiveness of ceftibuten or a pharmaceutically acceptable derivative thereof in a subject, said method comprising co-administering ceftibuten or a pharmaceutically acceptable derivative thereof, with clavulanic acid or a pharmaceutically acceptable derivative thereof, wherein the weight ratio of clavulanic acid or a pharmaceutically effective amount of, to ceftibuten or a pharmaceutically acceptable derivative in the composition is from about 1:8 to about 8:1.

In some other embodiments, there is provided a method for increasing antibacterial effectiveness of ceftibuten or a pharmaceutically acceptable derivative thereof in a subject, said method comprising co-administering ceftibuten or a pharmaceutically acceptable derivative thereof, with clavulanic acid or a pharmaceutically acceptable derivative thereof, wherein the amount of clavulanic acid or a pharmaceutically acceptable derivative thereof is from about 0.25 gram to about 4 gram per gram of ceftibuten or a pharmaceutically acceptable derivative thereof.

A wide variety of bacterial infections can be treated or prevented using compositions and methods according to the invention. Typical, non-limiting examples of bacterial infections that can be treated or prevented using methods and/or pharmaceutical compositions according to the invention include E. coli infections, Yersinia pestis (pneumonic plague), staphylococcal infection, mycobacteria infection, bacterial pneumonia, Shigella dysentery, Serratia infections, Candida infections, Cryptococcal infection, anthrax, tuberculosis or infections caused by Pseudomonas aeruginosa, Acinetobacter baumannii or methicillin resistant Staphylococcus aureus (MRSA) etc.

The pharmaceutical compositions and methods according to the invention are useful in treatment or prevention of several infections, including for example, skin and soft tissue infections, febrile neutropenia, urinary tract infection, intraabdominal infections, respiratory tract infections, pneumonia (nosocomial), bacteremia meningitis, surgical infections and the like.

In some embodiments, pharmaceutical compositions and methods according to the invention are used in treatment or prevention of infections caused by resistant bacteria. In some other embodiments, the compositions and methods according to the invention are used in treatment or prevention of infections caused by bacteria producing one or more beta-lactamase enzymes.

In general, the pharmaceutical compositions and methods disclosed herein are also effective in preventing or treating infections caused by bacteria that are considered to be less or not susceptible to one or more of known antibacterial agents or their known compositions. Some non-limiting examples of such bacteria known to have developed resistance to various antibacterial agents include Acinetobacter, Escherichia coli, Pseudomonas aeruginosa, Staphylococcus aureus, Enterobacter, Klebsiella, Citrobacter and a like.

In some embodiments, the pharmaceutical compositions and methods disclosed herein are also effective in preventing or treating infections caused by gram negative bacteria. In some embodiments, the pharmaceutical compositions and methods according to invention are also effective in preventing or treating infections caused by multi-drug resistant gram negative bacteria.

In some embodiments, pharmaceutical compositions and methods according to the invention are used for treatment or prevention of bacterial infections caused by strains expressing highly resistant Klebsiella pneumonia carbapenemase (KPC) beta-lactamase enzymes or outer membrane porin down (OMP) regulations that confer resistance to carbapenems.

### EXAMPLES

The following examples illustrate the embodiments of the invention that are presently best known. However, it is to be understood that the following are only exemplary or illustrative of the application of the principles of the present invention. Numerous modifications and alternative compositions, methods and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

The antibacterial activity of combinations according to the invention was investigated against various bacterial strains. In a typical study, minimum inhibitory concentrations (MICs) were determined using Muller Hinton Agar (MHA) (BD, USA) according to Clinical and Laboratory Standards Institute (CLSI) recommendations, (Clinical and Laboratory Standards Institute (CLSI), Performance Standards for Antimicrobial Susceptibility Testing, 20^{th} Informational Supplement, M 100-S20, Volume 30, No. 1, 2010). In short, the inocula were adjusted to deliver about 104 colony forming units (CFU) per spot with a multipoint inoculator (Applied Quality Services, UK). The plates were pored with doubling concentration range of the test combinations according to invention containing MHA. The plates were inoculated and were incubated at 35° C. for 18 hours. MICs were read as the lowest concentration of drug that completely inhibited bacterial growth.

The results of antibacterial activity of combination according to present invention against highly resistant gram negative bacterial strains are given in Table 1. As may be seen from the data in Table 1, the MIC values of ceftibuten significantly reduced in the presence of clavulanic acid. The data also reveals that combination of ceftibuten and clavulanic acid exhibited potent synergistic antibacterial activity, which was not observed for other tested combinations (ceftibuten and tazobactam; ceftibuten and sulbactam; cefixime and clavulanic acid; cefpodoxime and clavulanic acid; cefdinir and clavulanic acid).

The results of Table 1, clearly and surprisingly demonstrate that compositions according to the present invention exhibit synergistic antibacterial activity against bacteria that produce one or more beta-lactamase enzymes. Therefore, the combination of ceftibuten with clavulanic acid in specific amounts has tremendous beneficial effects in inhibiting highly resistant gram negative bacteria including those producing extended spectrum beta-lactamase enzymes.

### Example 2

Manufacturing Procedure:

The potassium clavulanate, ceftibuten dihydrate, microcrystalline cellulose, crosscarmellose sodium were weighed, sifted, and mixed in a Rapid Mixer Granulator. The above mass was granulated by spraying aqueous solution of povidone. The granules were dried in a fluidized bed drier, sifted and milled. The resulting granules were blended with sifted microcrystalline cellulose, crosscarmellose sodium, talc and magnesium stearate. The lubricated granules were compressed into tablets using suitable tooling. The tablets were coated with aqueous dispersion of opadry. The composition is shown in Table 2.

TABLE 1
Antibacterial activity of various antibacterial combinations against highly resistant gram negative bacterial strains.

| | | | | | MIC (μg/ml) of | | MIC (μg/ml) of | | MIC (μg/ml) of | | |
|---|---|---|---|---|---|---|---|---|---|---|---|
| | | | | | Cefixime in | | Cefpodoxime in | | Cefdinir in | | |
| | MIC (μg/ml) of Ceftibuten in presence of | | | | presence of | | presence of | | presence of | | MIC |
| | Control | Clavulanic | Tazo- | Sul- | Control | Clavulanic | Control | Clavulanic | Control | Clavulanic | (μg/ml) |
| Strains | (Ceftibuten | acid | bactam | bactam | (Cefixime | acid | (Cefpodoxime | acid | (Cefdinir | acid | of |
| (Genotype) | alone) | (4 μg/ml) | (4 μg/ml) | (4 μg/ml) | alone) | (4 μg/ml) | alone) | (4 μg/ml) | alone) | (4 μg/ml) | Imipenem |
| Klebsiella | 16 | 4 | 8 | 8 | >32 | 16 | >32 | >32 | >32 | >32 | 16 |
| H 515 | | | | | | | | | | | |
| (KPC, class | | | | | | | | | | | |
| A) | | | | | | | | | | | |
| Klebsiella | 32 | 4 | 16 | 8 | >32 | 16 | >32 | >32 | >32 | >32 | >32 |
| H 520 | | | | | | | | | | | |
| (KPC, class | | | | | | | | | | | |
| A) | | | | | | | | | | | |
| Klebsiella | 32 | 4 | 16 | 16 | >32 | 16 | >32 | >32 | >32 | >32 | >32 |
| H 521 | | | | | | | | | | | |
| (KPC, class | | | | | | | | | | | |
| A) | | | | | | | | | | | |
| Klebsiella | 16 | 4 | 8 | 4 | >32 | 16 | >32 | >32 | >32 | >32 | >32 |
| H 523 | | | | | | | | | | | |
| (KPC, class | | | | | | | | | | | |
| A) | | | | | | | | | | | |
| Klebsiella | >32 | 8 | 32 | 32 | >32 | >32 | >32 | >32 | >32 | >32 | 4 |
| J 101 | | | | | | | | | | | |
| (TEM, SHV, | | | | | | | | | | | |
| OMP) | | | | | | | | | | | |
| Klebsiella | 32 | 8 | 32 | 32 | >32 | >32 | >32 | >32 | >32 | >32 | 8 |
| J 102 | | | | | | | | | | | |
| (TEM, SHV, | | | | | | | | | | | |
| OMP) | | | | | | | | | | | |
| Klebsiella | 32 | 0.12 | 32 | 32 | >32 | 0.5 | >32 | 4 | >32 | 0.5 | 2 |
| J 117 | | | | | | | | | | | |
| (SHV, OMP) | | | | | | | | | | | |
| Klebsiella | >32 | 4 | >32 | >32 | >32 | 16 | >32 | 32 | >32 | 16 | 4 |
| J 130 | | | | | | | | | | | |
| (SHY, OMP) | | | | | | | | | | | |
| NCTC KP | 0.25 | 0.12 | 0.25 | 0.25 | 0.25 | 0.25 | 4 | 1 | 32 | 32 | 2 |
| 13442 | | | | | | | | | | | |
| (OXA-48) | | | | | | | | | | | |
| KP S 440 | 16 | 2 | 4 | 4 | >32 | 16 | >32 | >32 | >32 | >32 | 2 |
| (OXA 181, | | | | | | | | | | | |
| SHV, TEM, | | | | | | | | | | | |
| CTX-M 1) | | | | | | | | | | | |
| KP S 691 | 8 | 1 | 2 | 2 | >32 | 32 | >32 | >32 | >32 | >32 | 2 |
| (OXA 181, | | | | | | | | | | | |
| SHV, TEM, | | | | | | | | | | | |
| CTX-M 1) | | | | | | | | | | | |

Note:
Clavulanic acid, Tazobactam, and Sulbactam when used alone were inactive with MICs of >32 μg/ml

TABLE 2
Pharmaceutical compositions according to the invention

| | | mg/Tablet | |
|---|---|---|---|
| | | Formu- | Formu- |
| Sr. | Ingredients | lation 1 | lation 2 |

INTRAGRANULAR

| | | | |
|---|---|---|---|
| 1 | Potassium Clavulanate | 238.25 | 238.25 |
| 2 | Ceftibuten dihydrate | 208.40 | 416.80 |
| 3 | Microcrystalline Cellulose (Avicel PH | 80.0 | 160.0 |
| | 101) | | |
| 4 | Croscarmesllose Sodium (Ac-Di-Sol) | 7.0 | 14.0 |
| 5 | Povidone K30 (Kollidone K30) | 8.75 | 17.50 |
| 6 | Purified water USP | q.s. | q.s. |

EXTRAGRANULAR

| | | | |
|---|---|---|---|
| 7 | Microcrystalline cellulose (Avicel PH | 31.25 | 62.50 |
| | 102) | | |
| 8 | Croscarmesllose Sodium (Ac-Di-Sol) | 13.0 | 26.0 |
| 9 | Talc | 3.50 | 7.00 |
| 10 | Magnesium stearate | 3.0 | 6.0 |

| | | |
|---|---|---|
| CORE TABLET (mg) | 596.65 | 955.05 |

FILM COATING

| | | | |
|---|---|---|---|
| 11 | Opadry Yellow (03B28796) | 10.5 | 21.0 |
| 12 | Purified Water USP | q.s. | q.s. |

| | | |
|---|---|---|
| Total (Coated Tablet Weight) mg | 607.15 | 976.05 |

1. A pharmaceutical composition comprising synergistically effective amount of: (a) ceftibuten or a pharmaceutically acceptable derivative thereof, and (b) clavulanic acid or a pharmaceutically acceptable derivative thereof.

2. A pharmaceutical composition according to claim 1, wherein the weight ratio of clavulanic acid or a pharmaceutically acceptable derivative thereof, to ceftibuten or a pharmaceutically acceptable derivative thereof in the composition is from about 1:8 to about 8:1.

3. A pharmaceutical composition consisting of synergistically effective amount of: (a) ceftibuten or a pharmaceutically acceptable derivative thereof, and (b) clavulanic acid or a pharmaceutically acceptable derivative thereof as active ingredients; wherein the weight ratio of clavulanic acid or a pharmaceutically acceptable derivative thereof, to ceftibuten or a pharmaceutically acceptable derivative thereof in the composition is from about 1:8 to about 8:1.

4. A pharmaceutical composition according to any one of claims 1 to 3, wherein clavulanic acid or a pharmaceutically acceptable derivative thereof in the composition is present in an amount from about 0.25 gram to about 4 gram per gram of ceftibuten or a pharmaceutically acceptable derivative thereof.

5. A pharmaceutical composition according to any of the claims 1 to 4, comprising about 0.1 gram to about 10 gram of ceftibuten.

6. A pharmaceutical composition according to any of the claims 1 to 4, comprising about 0.1 gram to about 10 gram of clavulanic acid.

7. A pharmaceutical composition according to any of the claims 1 to 4, comprising: (a) ceftibuten or a pharmaceutically acceptable derivative thereof, and (b) clavulanic acid or a pharmaceutically acceptable derivative thereof, in any one of following amounts:
(i) about 0.2 gram of ceftibuten or a pharmaceutically acceptable derivative thereof and about 0.4 gram of clavulanic acid or a pharmaceutically acceptable derivative thereof;
(ii) about 0.2 gram of ceftibuten or a pharmaceutically acceptable derivative thereof and about 0.2 gram of clavulanic acid or a pharmaceutically acceptable derivative thereof;
(iii) about 0.2 gram of ceftibuten or a pharmaceutically acceptable derivative thereof and about 0.125 gram of clavulanic acid or a pharmaceutically acceptable derivative thereof;
(iv) about 0.2 gram of ceftibuten or a pharmaceutically acceptable derivative thereof and about 0.0625 gram of clavulanic acid or a pharmaceutically acceptable derivative thereof;
(v) about 0.4 gram of ceftibuten or a pharmaceutically acceptable derivative thereof and about 0.8 gram of clavulanic acid or a pharmaceutically acceptable derivative thereof;
(vi) about 0.4 gram of ceftibuten or a pharmaceutically acceptable derivative thereof and about 0.4 gram of clavulanic acid or a pharmaceutically acceptable derivative thereof;
(vii) about 0.4 gram of ceftibuten or a pharmaceutically acceptable derivative thereof and about 0.2 gram of clavulanic acid or a pharmaceutically acceptable derivative thereof;
(viii) about 0.4 gram of ceftibuten or a pharmaceutically acceptable derivative thereof and about 0.125 gram of clavulanic acid or a pharmaceutically acceptable derivative thereof;
(ix) about 0.4 gram of ceftibuten or a pharmaceutically acceptable derivative thereof and about 0.0625 gram of clavulanic acid or a pharmaceutically acceptable derivative thereof;
(x) about 0.5 gram of ceftibuten or a pharmaceutically acceptable derivative thereof and about 0.2 gram of clavulanic acid or a pharmaceutically acceptable derivative thereof;
(xi) about 0.5 gram of ceftibuten or a pharmaceutically acceptable derivative thereof and about 0.125 gram of clavulanic acid or a pharmaceutically acceptable derivative thereof; or
(xii) about 0.5 gram of ceftibuten or a pharmaceutically acceptable derivative thereof and about 0.0625 gram of clavulanic acid or a pharmaceutically acceptable derivative thereof.

8. A pharmaceutical composition according to any of the claims 1 to 7, wherein ceftibuten is present as ceftibuten dihydrate.

9. A pharmaceutical composition according to any of the claims 1 to 7, wherein clavulanic acid is present as potassium clavulanate.

10. A pharmaceutical composition according to any one of the claims 1 to 7, further comprising one or more pharmaceutically acceptable excipients or carriers.

11. A pharmaceutical composition according to any of the claims 1 to 10, wherein the composition is formulated into a dosage form such that ceftibuten or a pharmaceutically acceptable derivative thereof, and clavulanic acid or a pharmaceutically acceptable derivative thereof are present in the composition as admixture or as separate components.

12. A pharmaceutical composition according to any of the claims 1 to 11, wherein the composition is in the form of a powder or a solution.

13. A pharmaceutical composition according to claim 12, wherein the composition is in the form of a powder or a solution that can be reconstituted by addition of a compatible reconstitution diluent for oral or parenteral administration.

14. A pharmaceutical composition according to any of the claims 1 to 12, wherein the composition is formulated into a dosage form suitable for oral administration.

15. A pharmaceutical composition according to any of the claims 1 to 14, for use in treatment or prevention of a bacterial infection.

16. A method of treating or preventing a bacterial infection in a subject, said method comprising administering to said subject an effective amount of a pharmaceutical composition according to any of the claims 1 to 14.

17. A method of treating or preventing a bacterial infection in a subject, said method comprising administering to said subject synergistically effective amount of: (a) ceftibuten or a pharmaceutically acceptable derivative thereof, and (b) clavulanic acid or a pharmaceutically acceptable derivative thereof.

18. A method of treating or preventing a bacterial infection according to claim 17, wherein amount of clavulanic acid or a pharmaceutically acceptable derivative thereof, and ceftibuten or a pharmaceutically acceptable derivative thereof administered are in weight ratio of about 1:8 to about 8:1.

19. A method according to any one of the claims 17 or 18, wherein clavulanic acid or a pharmaceutically acceptable derivative thereof is administered in an amount from about 0.25 gram to about 4 gram per gram of ceftibuten or a pharmaceutically acceptable derivative thereof.

20. A method according to any of the claims 17 to 19, wherein clavulanic acid or a pharmaceutically acceptable derivative thereof and ceftibuten or a pharmaceutically acceptable derivative thereof are administered in any of the following amounts:
(i) about 0.2 gram of ceftibuten or a pharmaceutically acceptable derivative thereof and about 0.4 gram of clavulanic acid or a pharmaceutically acceptable derivative thereof;
(ii) about 0.2 gram of ceftibuten or a pharmaceutically acceptable derivative thereof and about 0.2 gram of clavulanic acid or a pharmaceutically acceptable derivative thereof;
(iii) about 0.2 gram of ceftibuten or a pharmaceutically acceptable derivative thereof and about 0.125 gram of clavulanic acid or a pharmaceutically acceptable derivative thereof;
(iv) about 0.2 gram of ceftibuten or a pharmaceutically acceptable derivative thereof and about 0.0625 gram of clavulanic acid or a pharmaceutically acceptable derivative thereof;
(v) about 0.4 gram of ceftibuten or a pharmaceutically acceptable derivative thereof and about 0.8 gram of clavulanic acid or a pharmaceutically acceptable derivative thereof;
(vi) about 0.4 gram of ceftibuten or a pharmaceutically acceptable derivative thereof and about 0.4 gram of clavulanic acid or a pharmaceutically acceptable derivative thereof;
(vii) about 0.4 gram of ceftibuten or a pharmaceutically acceptable derivative thereof and about 0.2 gram of clavulanic acid or a pharmaceutically acceptable derivative thereof;
(viii) about 0.4 gram of ceftibuten or a pharmaceutically acceptable derivative thereof and about 0.125 gram of clavulanic acid or a pharmaceutically acceptable derivative thereof;
(ix) about 0.4 gram of ceftibuten or a pharmaceutically acceptable derivative thereof and about 0.0625 gram of clavulanic acid or a pharmaceutically acceptable derivative thereof;
(x) about 0.5 gram of ceftibuten or a pharmaceutically acceptable derivative thereof and about 0.2 gram of clavulanic acid or a pharmaceutically acceptable derivative thereof;
(xi) about 0.5 gram of ceftibuten or a pharmaceutically acceptable derivative thereof and about 0.125 gram of clavulanic acid or a pharmaceutically acceptable derivative thereof; or
(xii) about 0.5 gram of ceftibuten or a pharmaceutically acceptable derivative thereof and about 0.0625 gram of clavulanic acid or a pharmaceutically acceptable derivative thereof.

21. The method according to any of the claims 17 to 20, wherein ceftibuten or a pharmaceutically acceptable derivative thereof is administered before, after or simultaneously with the administration of clavulanic acid or a pharmaceutically acceptable derivative thereof.

22. A method of increasing antibacterial effectiveness of ceftibuten or a pharmaceutically acceptable derivative thereof in a subject, said method comprising co-administering ceftibuten or a pharmaceutically acceptable derivative thereof, with clavulanic acid or a pharmaceutically acceptable derivative thereof.

23. A method according to claim 22, wherein amount of clavulanic acid or a pharmaceutically acceptable derivative thereof, and ceftibuten or a pharmaceutically acceptable derivative thereof administered are in weight ratio of about 1:8 to about 8:1.

24. A method according to any one of claims 22 or 23, wherein clavulanic acid or a pharmaceutically acceptable derivative thereof is administered in an amount from about 0.25 gram to about 4 gram per gram of ceftibuten or a pharmaceutically acceptable derivative thereof.
